(19) United States
(12) Patent Application Publication
(10) Pub. No.: US 2026/0272516 A1
(43) Pub. Date: Sep. 17, 2026

(54) SPINAL FIXATION SYSTEM WITH VISUALIZATION APERTURE AND SLIDING PIN ENGAGEMENT FOR VERTEBRAL STABILIZATION

(71) Applicant: Daniel Martin Harwell, Tulsa, OK (US)

(72) Inventor: Daniel Martin Harwell, Tulsa, OK (US)

(21) Appl. No.: 19/076,760

(22) Filed: Mar. 11, 2025

(51) Int. Cl. A61B 17/70; A61B 17/68; A61F 2/44

(52) U.S. Cl. CPC A61B 17/7059; A61F 2/4455; A61B 2017/681

(57) ABSTRACT

A spinal fixation system includes implantable plates designed to enhance anterior lumbar surgical procedures through optimized vertebral stabilization architecture. The system employs advanced plate geometries including: a rectangular plate configuration featuring strategically positioned bone screw holes for robust vertebral fixation, an H-shaped plate design enabling efficient multi-level construct assembly, and a D-shaped plate geometry facilitating acentric mounting for complex spinal alignments. Each plate configuration incorporates precision-engineered visualization apertures positioned to enable direct operative site observation while maintaining structural integrity. The plates feature spherical bone screw interfaces enabling variable angle screw placement to accommodate patient-specific anatomy. The system's multi-level capability is achieved through complementary plate geometries that create parallel visualization corridors when assembled in series.

### CROSS REFERENCE TO RELATED APPLICATIONS

N/A.

### TECHNICAL FIELD

The present disclosure relates generally to spinal fixation devices, and more particularly to an implantable spinal stabilization system featuring a plate with integrated visualization and engagement mechanisms, designed to improve surgical accuracy and facilitate multi-level vertebral fusion procedures.

### SUMMARY

According to some embodiments the present disclosure is directed to a spinal fixation device. The spinal fixation device also includes a plate configured for attachment to a first vertebral body and a second vertebral body, the plate may include: a first arm configured to extend upward from a central portion; a second arm configured to extend downward from the central portion; at least one bone screw hole in each of the first and second arms, each bone screw hole configured to receive a bone screw for securing the arms to the respective vertebral bodies; and a visualization aperture extending through the central portion.

Implementations may include one or more of the following features. The device may include: a third arm configured to extend upward from the central portion in parallel with the first arm; and a fourth arm configured to extend downward from the central portion in parallel with the second arm; where each of the third and fourth arms may include at least one bone screw hole configured to receive a bone screw. Colon>the first and third arms define a first space therebetween configured to accommodate a temporary distraction pin; and the second and fourth arms define a second space therebetween configured to accommodate a temporary distraction pin. The visualization aperture is positioned centrally between the first, second, third and fourth arms. The visualization aperture is positioned between the arms and dimensioned to maintain structural integrity of the plate. Colon>the visualization aperture is positioned between the arms and dimensioned to accommodate surgical instruments while maintaining structural integrity of the plate. The first and third arms extend upward at divergent angles relative to each other. The device may include an interbody cage positioned between the first vertebral body and the second vertebral body. Each bone screw hole may include a spherical seat portion configured to engage a complementary spherical head of a bone screw. Implementations of the described techniques may include hardware, a method or process, or computer software on a computer-accessible medium.

According to some embodiments the present disclosure is directed to a spinal fixation device. The spinal fixation device also includes a d-shaped plate configured for attachment between vertebral bodies, the plate may include a curved edge having a substantially semi-circular profile. The device also includes a straight edge intersecting the curved portion; at least two bone screw holes positioned along the curved edge. The device also includes a visualization aperture extending through the plate between the curved edge and the straight edge.

Implementations may include one or more of the following features. The device where each bone screw hole may include a spherical seat portion configured to engage a complementary spherical head of a bone screw. The visualization aperture is dimensioned to maintain structural integrity of the plate.

According to some embodiments the present disclosure is directed to a dynamic spinal stabilization device. The dynamic spinal stabilization device also includes first and second arcuate members extending between a first vertebral body and a second vertebral body. The device also includes each arcuate member may include: a pivotal mounting interface at an inferior end configured for rotatable attachment to the first vertebral body, a track formation at a superior end configured to slidably receive a bone screw shaft, where the first and second arcuate members are configured to enable controlled flexion between the vertebral bodies through coordinated pivotal movement at the inferior ends and translation of bone screws within the track formations.

Implementations may include one or more of the following features. The device where each track formation may include: a channel having a width dimensioned to guide the bone screw shaft during anterior-posterior translation while preventing lateral deviation. The arcuate members extend in opposing curved trajectories. The first and second arcuate members are configured to maintain alignment between the first vertebral body and the second vertebral body throughout a range of motion. The device may include an interbody fusion device positioned between the first vertebral body and second vertebral body. Each bone screw may include a spherical head configured to enable variable angle placement. The track formations are positioned to generate a predetermined motion pathway during spinal flexion.

### BRIEF DESCRIPTION OF THE DRAWINGS

FIG. 1 illustrates a first embodiment of a spinal fixation system including a plate secured to first and second vertebral bodies.

FIG. 2 depicts an enlarged perspective view of the spinal fixation system of FIG. 1, showing the relationship between plate components and vertebral bodies.

FIG. 3 presents a detailed view of operative engagement between plate elements and vertebral structures.

FIG. 4 illustrates a multi-level spinal fixation assembly utilizing first and second plates secured across multiple vertebral levels.

FIG. 5 provides a frontal view of a spinal fixation system incorporating a D-shaped plate configuration.

FIG. 6 depicts the D-shaped plate configuration of FIG. 5 in a multi-level arrangement.

FIG. 7 shows a spinal fixation system utilizing a rectangular plate configuration with opposed lateral arms.

FIG. 8 illustrates operative engagement of the rectangular plate configuration.

FIG. 9 illustrates a spinal fixation system incorporating dynamic stabilization features through arcuate members with superior tracking mechanisms and inferior pivotal interfaces.

FIG. 10 is a cross section of a portion of the device illustrated in FIG. 9, taken about section line 10-10.

### DETAILED DESCRIPTION OF EXAMPLE EMBODIMENTS

### Overview

The present technology relates to spinal fixation systems, specifically implant plates designed to enhance anterior lumbar surgical procedures by providing improved vertebral stabilization through optimized plate geometries and enhanced surgical visualization. These systems address challenges in current designs, such as operative site access, construct stability, and efficient management of multi-level vertebral procedures. The inventive plates incorporate strategic visualization apertures and specialized geometric configurations that enable precise surgical placement while accommodating temporary distraction pins during vertebral manipulation.

Some embodiments include a rectangular plate configuration, an H-shaped plate, and a D-shaped plate, each engineered for specific surgical applications. The rectangular configuration features two pairs of bone screw holes connected by a central portion incorporating a visualization aperture. This architecture provides robust fixation to adjacent vertebral bodies while maintaining direct operative site observation. The H-shaped plate includes laterally opposed arms at superior and inferior ends, with spaces between the arms configured to accommodate temporary distraction pins while enabling efficient plate placement. This embodiment supports secure alignment for complex spinal stabilization needs. The D-shaped plate combines a curved portion having a substantially semi-circular profile with a straight portion, specifically engineered to facilitate multi-level applications by ensuring appropriate anatomical offsets and maintaining alignment between vertebral bodies.

Each plate configuration is engineered to provide independent functionality or integration within multi-level constructs. Bone screw holes are strategically positioned to achieve optimal fixation to vertebral bodies, with spherical seat portions enabling variable angle screw placement to accommodate patient-specific anatomy. The visualization apertures are precisely dimensioned and positioned to maintain structural integrity while providing direct observation access throughout the surgical procedure. This comprehensive approach enables enhanced surgical precision during implantation and facilitates post-operative fusion assessment. The system thereby addresses fundamental challenges in current spinal fixation technology through advanced plate geometries optimized for surgical efficiency and construct stability.

### Example Embodiments

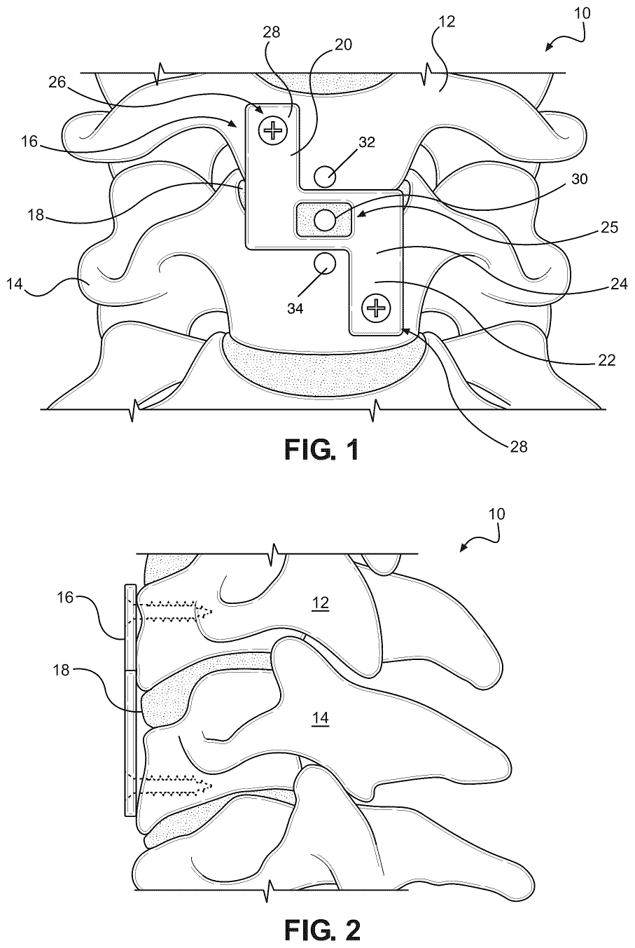

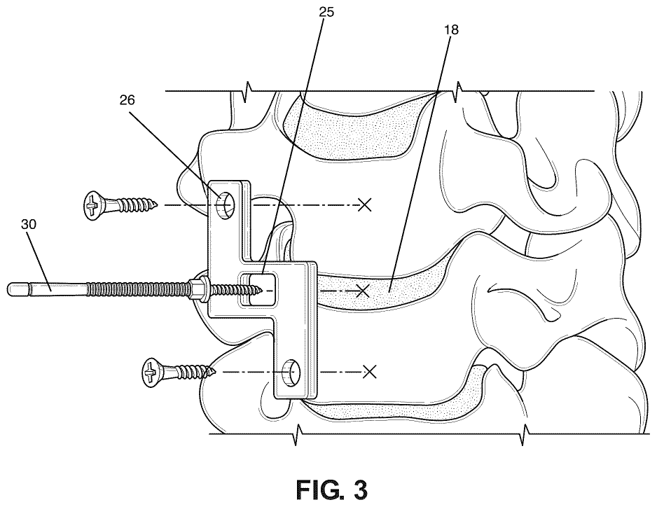

FIGS. 1-3 depict a spinal fixation system 10, which is configured to engage a first vertebral body 12 and a second vertebral body 14. The system is shown in its application to two adjacent vertebrae for illustrative purposes but is adaptable for use at multiple anatomical levels. Spinal fixation system 10 includes a fixation plate 16 and an interbody cage 18, which may be utilized independently or together based on surgical preferences or patient-specific requirements.

Fixation plate 16 comprises a first arm portion 20 and a second arm portion 22, extending longitudinally in opposite directions from a central portion 24. The central portion 24 is characterized by a visualization aperture 25 that extends completely through the plate. This aperture provides a direct visual corridor to the interbody cage 18 and the surrounding operative site during implantation and subsequent fusion assessments. The visualization aperture 25 has an elongated, oval shape, designed to balance sufficient surgical access with the structural integrity of the fixation plate 16.

Bone screw holes 26 are located at the lateral ends of both the first arm portion 20 and the second arm portion 22. Each bone screw hole 26 features a spherical seat for engagement with the spherical head of a bone screw 28, enabling a variable-angle placement that allows surgeons to optimize screw trajectories based on anatomical considerations. As shown, the screws 28 are depicted with threaded shanks designed to achieve strong purchase within the bone.

The central portion 24 includes offset geometry, where the arm portions 20 and 22 are positioned at varying anterior-posterior depths, corresponding to the natural lordotic angle of the vertebrae. The visualization aperture 25 allows for the projection of a first distraction pin 30 from the interbody cage 18 during the procedure, creating an integrated visualization and access corridor that extends from the anterior surface of the fixation plate 16 to the operative site. This configuration facilitates precise observation and alignment of the interbody cage 18 during implantation.

Additional temporary distraction pins 32 and 34 may be inserted into the first vertebral body 12 and the second vertebral body 14 to maintain the appropriate disc space height during surgery. The fixation plate's overall geometry is adapted to ensure stability during attachment and accommodates the natural curvature of the spine. The plate's outer edges are contoured, minimizing interference with adjacent anatomical structures and reducing the risk of soft tissue irritation.

The interbody cage 18, as depicted, has a generally rectangular shape with tapered edges, aiding in ease of insertion and optimal positioning between the vertebral bodies. The cage features a textured surface or integrated teeth (not shown in detail but implied in the drawings) to enhance fixation and stability once inserted. In some embodiments, the fixation plate 16 may be fastened to the interbody cage 18 through a threaded fastener or screw, which is installed after the distraction pin 30 is removed, securing the two components into a unified construct. Alternatively, the fixation plate 16 and interbody cage 18 may be installed separately, with the plate serving as an independent stabilization mechanism when the interbody cage 18 is not required.

When coupled, the fixation plate 16 and interbody cage 18 form a robust stabilization system, designed to maintain the integrity of the spinal column during fusion. These components can also be adapted for use with additional instrumentation, as indicated by the modular design visible in the drawings.

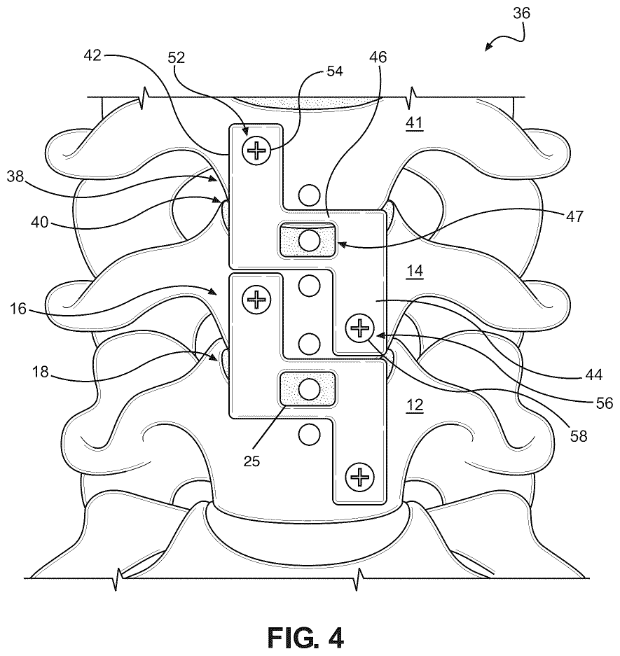

FIG. 4 illustrates a multi-level spinal fixation assembly 36, designed to stabilize two adjacent levels of the spine. The assembly consists of a first fixation plate 16 and a second fixation plate 38, each configured for attachment to respective interbody cages: the first interbody cage 18 and the second interbody cage 40. These plates and cages work together to provide secure stabilization while allowing independent assessment of fusion progress at each spinal level.

The second fixation plate 38 shares structural similarities with the first fixation plate 16, featuring a first arm portion 42 and a second arm portion 44 that extend laterally in opposite directions from a central portion 46. The central portion 46 includes a visualization aperture 47 that extends entirely through the plate. This aperture facilitates direct visualization of the operative site, enabling precise placement of the second interbody cage 40 and subsequent monitoring of fusion development. The aperture is elongated, offering a balance between visual access and structural integrity while permitting the use of surgical instruments.

Each arm portion 42 and 44 of the second fixation plate 38 includes a set of bone screw holes, labeled 52 and 56, positioned at their respective lateral ends. These screw holes feature spherical seats designed to accommodate the spherical heads of bone screws 54 and 58. This configuration allows for variable-angle screw placement, enabling the surgeon to optimize the screw trajectories for secure fixation to the vertebral bodies while accommodating anatomical variances.

When assembled in a multi-level configuration, the first fixation plate 16 engages the first vertebral body 12 and second vertebral body 14, while the second fixation plate 38 engages the second vertebral body 14 and third vertebral body 41. The visualization apertures 25 and 47 of the two fixation plates align to create parallel visualization corridors. These corridors enable independent observation of the fusion process at each level while maintaining the structural integrity required for multi-level spinal stabilization. The central portions 24 and 46 of the fixation plates are offset to account for the natural lordotic curvature of the spine, ensuring that both fixation plates achieve the proper positioning and screw trajectory for optimal stability and functionality.

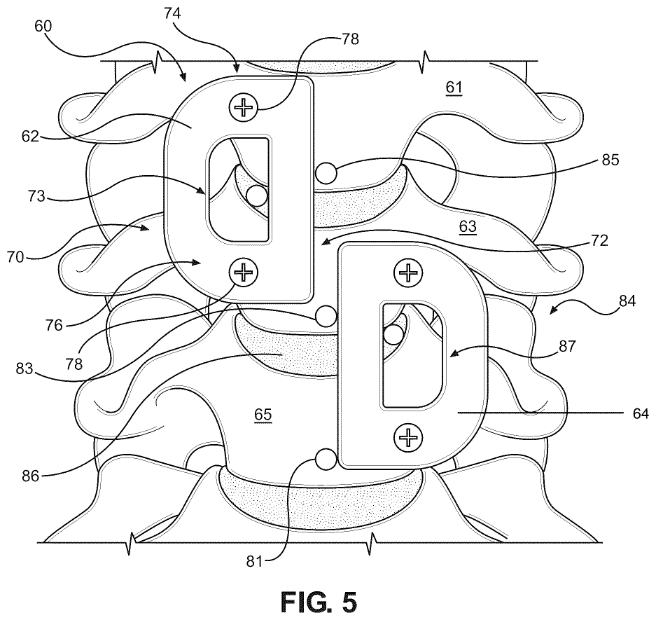

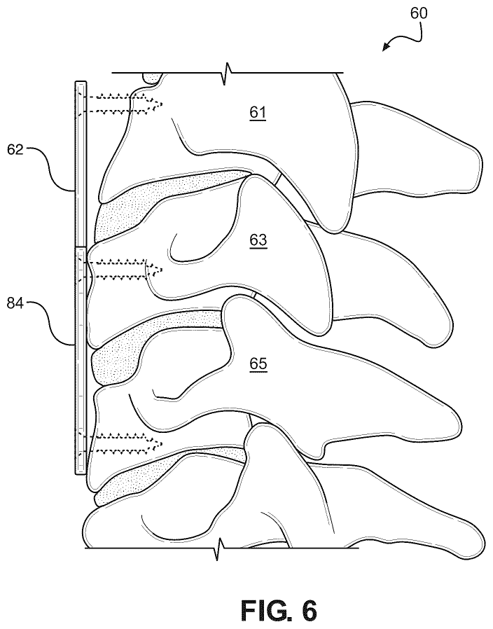

FIGS. 5 and 6 illustrate a spinal fixation system 60 featuring a distinctive D-shaped fixation plate 62, designed for stabilizing adjacent vertebral bodies while providing visual and mechanical access to the operative site. The D-shaped plate 62 is defined by a curved edge 70 with a semi-circular profile and a straight edge 72, creating its characteristic “D” shape. The plate 62 is configured to couple a first vertebral body 61 and a second vertebral body 63, providing robust stabilization during fusion procedures.

A visualization aperture 73 extends through the body of the D-shaped plate 62, strategically located between the curved edge 70 and the straight edge 72. This aperture is elongated and optimally positioned to provide direct visualization of the interbody cage 64 and surrounding anatomy, aiding in both initial placement and subsequent monitoring of fusion progress. The aperture's dimensions ensure adequate surgical instrument access without compromising the structural integrity of the plate.

The curved edge 70 of the D-shaped plate 62 includes two bone screw holes: a first hole 74 located near one end of the curve and a second hole 76 near the opposite end. These bone screw holes feature circular seats designed to accommodate spherical-headed bone screws 78, enabling variable-angle screw placement. This design ensures secure engagement with the vertebral bodies while allowing for adjustment based on the unique anatomical and surgical requirements of each procedure.

For multi-level spinal fixation applications, a second D-shaped plate 84 is introduced. This plate is similarly configured, with a visualization aperture 87 aligned to complement visualization aperture 73 in the first D-shaped plate 62. The alignment of these apertures creates a unified visualization corridor, enabling optimized surgical access and observation across multiple levels. The second D-shaped plate 84 is designed to engage a second interbody cage 86, coupling vertebral body 63 and a third vertebral body 65. Bone screws inserted through corresponding bone screw holes in the second D-shaped plate 84 provide secure fixation at the additional level.

The D-shaped plates 62 and 84 are designed for acentric mounting, which facilitates efficient stacking of the plates during multi-level procedures. This configuration ensures proper anatomical offsets, accommodating the natural lordotic curvature of the spine while maintaining accurate screw trajectories for stabilization. Temporary distraction pins 81, 83, and 85 can be used to manipulate the vertebral bodies during installation of the interbody cages 64 and 86. The D-shaped plates are versatile in their application: they can be coupled to their respective interbody cages before installation or after initial placement of the cages within the intervertebral space.

The integration of D-shaped plates 62 and 84 with interbody cages 64 and 86 forms a cohesive system that provides mechanical support, visualization capabilities, and flexibility for a variety of surgical approaches. This system is particularly effective in multi-level spinal fixation procedures, where independent yet aligned stabilization and observation are critical for achieving successful outcomes.

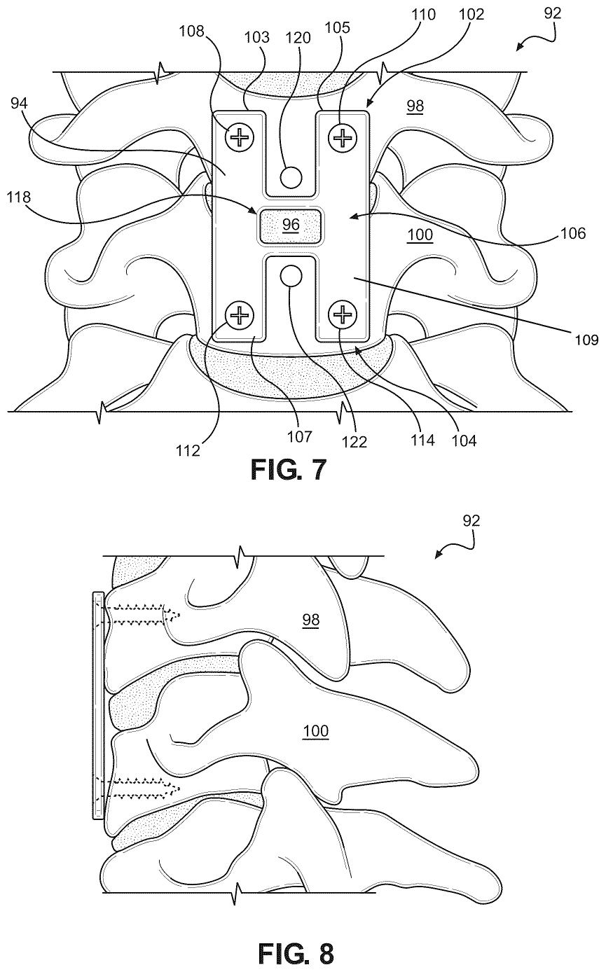

FIGS. 7 and 8 illustrate an “H-shaped” spinal fixation system 92, designed to provide robust stabilization for adjacent vertebral bodies. The system consists of a rectangular plate 94 and an interbody cage 96, working in tandem to facilitate spinal fusion. Rectangular plate 94 features a substantially rectangular body with distinct first and second end portions, 102 and 104, respectively, connected by a central portion 106.

The first end portion 102 is formed by two outwardly extending arms, 103 and 105, which create a U-shaped configuration at the plate's upper end. Each arm includes a lateral bone screw hole, 108 and 110, designed to accommodate bone screws with spherical heads. These spherical seats allow for variable-angle screw placement, enabling surgeons to achieve optimal engagement with vertebral bodies 98 and 100 based on anatomical and surgical needs. Similarly, the second end portion 104 features a pair of arms, 107 and 109, extending outward to form a mirror-image U-shaped configuration. Bone screw holes 112 and 114 are positioned on these arms to mirror the upper end, ensuring consistent fixation at both ends of the rectangular plate 94.

The central portion 106 of the plate incorporates a visualization aperture 118, centrally located and dimensioned to provide direct access to the interbody cage 96 and the surrounding operative site. This aperture is elongated and rectangular, offering a clear line of sight for precision during cage placement and subsequent monitoring of fusion progress. The aperture's size and positioning also accommodate surgical instruments, enabling seamless integration of tools for manipulation or assessment of the interbody cage.

The rectangular geometry of plate 94, in conjunction with its four-point fixation pattern, distributes forces evenly across the vertebral bodies 98 and 100. This configuration enhances stability, reducing the risk of localized stress concentrations and ensuring effective load transfer between the hardware and the spine.

A notable feature of the arms at both ends of the rectangular plate is the spacing between them, specifically designed to accommodate temporary distraction pins. Between arms 103 and 105 at the first end portion, a space is provided for distraction pin 120, while a similar space exists between arms 107 and 109 at the second end portion for distraction pin 122. These distraction pins serve to manipulate and maintain proper disc space height during the installation of the interbody cage 96 and the fixation system.

In this H-shaped configuration, the system combines the stability of a rectangular plate with the functional advantages of multiple fixation points and integrated visualization access. The structural symmetry and robust fixation design make this spinal fixation system particularly effective for addressing multi-level stabilization requirements or challenging anatomical scenarios, providing both mechanical support and enhanced surgical visualization.

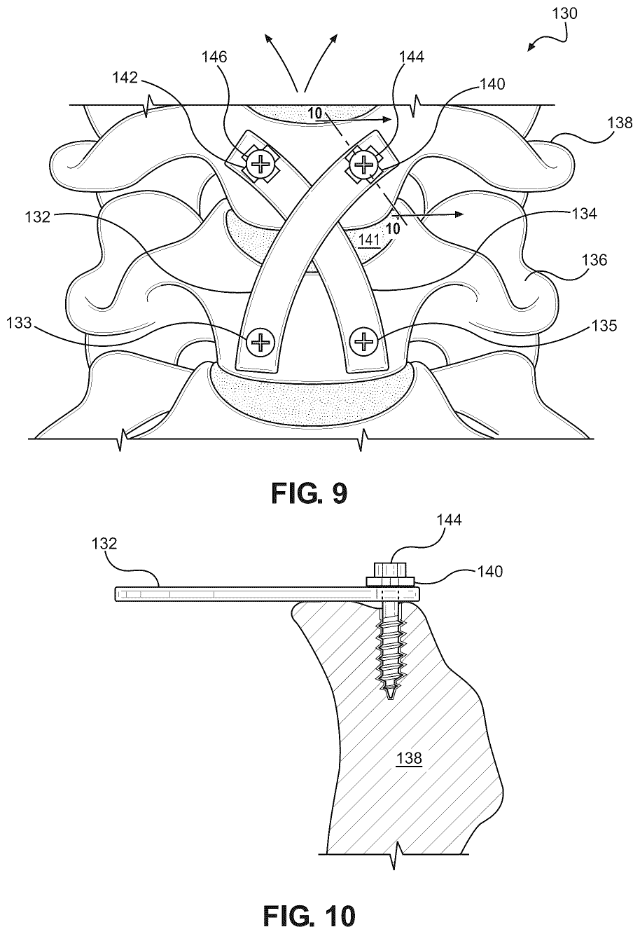

FIGS. 9 and 10 depict a dynamic spinal fixation system 130, designed to provide biomechanical stabilization while allowing controlled vertebral motion between the first vertebral body 136 and the second vertebral body 138. This system incorporates an interbody fusion device 141, strategically positioned between the vertebral bodies to maintain intervertebral spacing and promote osseointegration during the controlled motion protocols. The integration of stabilization and motion control makes this system particularly suitable for addressing both structural and biomechanical requirements in spinal treatments.

The system features dual arcuate members 132 and 134, each extending in opposing curved trajectories to form the core dynamic elements of the construct. These arcuate members are equipped with track formations 140 and 142, designed to guide the controlled translation of bone screws 144 and 146. The tracks are precisely dimensioned with tolerances and surface characteristics optimized to ensure smooth anterior-posterior translation of the bone screws along a predetermined pathway. This configuration minimizes lateral deviation and rotational instability, even under physiologic loading conditions. The track formations 140 and 142 provide both stability and flexibility, facilitating guided motion while maintaining the mechanical integrity of the construct.

The tracks 140 and 142 integrated into the arcuate members 132 and 134 may be configured with curved or straight profiles, depending on the desired biomechanical objectives of the spinal fixation system. A curved track design, for instance, can be tailored to promote controlled, physiologically accurate motion that mimics natural spinal kinematics during flexion and extension. Such curvature can guide bone screws 144 and 146 along a predetermined trajectory that aligns with the intervertebral anatomy, ensuring smooth and stable movement while preventing undesirable deviations or rotations. This adaptability in track geometry allows the system to accommodate various anatomical and clinical requirements, enhancing its effectiveness in providing dynamic stabilization while maintaining the structural integrity of the construct throughout its range of motion.

At their inferior ends, arcuate members 132 and 134 incorporate specialized mounting interfaces, identified as inferior mounting interfaces 133 and 135. These interfaces allow pivotal attachment to the first vertebral body 136, establishing primary fulcrum points. These fulcrum points work in coordination with the sliding bone screws, enabling the arcuate members to replicate natural spinal kinematics. This pivotal mechanism ensures balanced load distribution across the construct during dynamic movement, supporting the system's dual role of motion control and structural stabilization.

The bone screws 144 and 146 are engineered with dual functionalities. Their specialized geometric features enable secure fixation to the vertebral bodies while allowing controlled translation within the tracks 140 and 142. The screw shafts maintain stable engagement with the track formations, ensuring smooth anterior-posterior motion during spinal flexion and extension. This guided movement preserves the spatial relationship between the vertebral bodies and the interbody fusion device 141, ensuring consistent mechanical alignment throughout the motion arc.

The coordinated motion pathway established by the system is a result of the precise interaction between the track-guided screw translation and the inferior pivotal mounting points. The arcuate members 132 and 134 direct the second vertebral body 138 along a physiologically appropriate range of motion while simultaneously stabilizing the interbody fusion device 141. The system's architecture prevents excessive motion through the strategic geometry of the tracks, ensuring safe and controlled dynamic movement.

The spinal fixation systems described herein can be fabricated from a variety of biocompatible materials to ensure safety, durability, and mechanical integrity during and after implantation. Common materials include medical-grade titanium or titanium alloys (e.g., Ti-6Al-4V) due to their high strength-to-weight ratio, corrosion resistance, and excellent biocompatibility, which minimizes the risk of adverse reactions in the body. Alternatively, cobalt-chromium alloys may be employed for their superior wear resistance and rigidity, particularly in load-bearing applications. In some instances, components such as interbody cages may incorporate polyetheretherketone (PEEK), a radiolucent polymer that allows for post-operative imaging assessment while offering a degree of flexibility to complement the rigid metallic components. Surface treatments, such as plasma spraying or hydroxyapatite coatings, can be applied to metallic surfaces to enhance osseointegration and promote bone growth around the fixation devices. These material choices are designed to optimize performance, ensure long-term durability, and facilitate the healing process for patients undergoing spinal fusion or dynamic stabilization procedures.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

In this description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

1. A spinal fixation device, comprising:
a plate configured for attachment to a first vertebral body and a second vertebral body, the plate comprising:
a first arm configured to extend upward from a central portion;
a second arm configured to extend downward from the central portion;
at least one bone screw hole in each of the first and second arms, each bone screw hole configured to receive a bone screw for securing the arms to the respective vertebral bodies; and
a visualization aperture having an elongated shape extending through the central portion.

2. The device of claim 1, further comprising:
a third arm configured to extend upward from the central portion in parallel with the first arm; and
a fourth arm configured to extend downward from the central portion in parallel with the second arm;
wherein each of the third and fourth arms comprises at least one bone screw hole configured to receive a bone screw.

3. The device of claim 2, wherein the first and third arms define a first space therebetween configured to accommodate a temporary distraction pin; and the second and fourth arms define a second space therebetween configured to accommodate a temporary distraction pin.

4. The device of claim 2, wherein the visualization aperture is positioned centrally between the first, second, third and fourth arms.

5. The device of claim 2, wherein the visualization aperture is positioned between the arms and dimensioned to maintain structural integrity of the plate.

6. The device of claim 2, wherein the visualization aperture is positioned between the arms and dimensioned to accommodate surgical instruments while maintaining structural integrity of the plate.

7. The device of claim 1, further comprising a third arm configured to extend upward from the central portion, wherein the first arm and the third arm extend upward at divergent angles relative to each other.

8. The device of claim 1, further comprising an interbody cage positioned between the first vertebral body and the second vertebral body.

9. The device of claim 1, wherein each bone screw hole comprises a spherical seat portion configured to engage a complementary spherical head of a bone screw.

10. A spinal fixation device, comprising:
a D-shaped plate configured for attachment between vertebral bodies, the plate comprising:
a curved edge having a substantially semi-circular profile;
a straight edge intersecting the curved edge; at least two bone screw holes positioned along the curved edge; and
a visualization aperture extending through the plate between the curved edge and the straight edge.

11. The device of claim 10, wherein each bone screw hole comprises a spherical seat portion configured to engage a complementary spherical head of a bone screw.

12. The device of claim 11, wherein the visualization aperture is dimensioned to maintain structural integrity of the plate.

13. A dynamic spinal stabilization device, comprising:
first and second arcuate members extending between a first vertebral body and a second vertebral body;
each arcuate member further comprising:
a pivotal mounting interface at an inferior end configured for rotatable attachment to the first vertebral body;
a track formation at a superior end configured to slidably receive a bone screw shaft;
wherein the first and second arcuate members are configured to enable controlled flexion between the vertebral bodies through coordinated pivotal movement at the inferior ends and translation of bone screws within the track formations.

14. The device of claim 13, wherein each track formation comprises: a channel having a width dimensioned to guide the bone screw shaft during anterior-posterior translation while preventing lateral deviation.

15. The device of claim 13, wherein the arcuate members extend in opposing curved trajectories.

16. The device of claim 13, wherein the first and second arcuate members are configured to maintain alignment between the first vertebral body and the second vertebral body throughout a range of motion.

17. The device of claim 13, further comprising an interbody fusion device positioned between the first vertebral body and second vertebral body.

18. The device of claim 13, wherein each bone screw comprises a spherical head configured to enable variable angle placement.

19. The device of claim 13, wherein the track formations are positioned to generate a predetermined motion pathway during spinal flexion.
